(19) United States
(12) Patent Application Publication
(10) Pub. No.: US 2020/0041119 A1
(43) Pub. Date: Feb. 6, 2020

(54) Latent Heat and Crystalline Water Combination Power and Desalination Plant

(71) Applicant: Lane Asha Zabel, Coarsegold, CA (US)

(72) Inventor: Lane Asha Zabel, Coarsegold, CA (US)

(21) Appl. No.: 15/681,396

(22) Filed: Aug. 20, 2017

(51) Int. Cl. F22B 1/00; F28B 9/08; C07C 51/43

(52) U.S. Cl. CPC F22B 1/006; C02F 1/14; C07C 51/43; F28B 9/08

(57) ABSTRACT

(A) The inventions field is innovations in renewable energy production and desalinization. (B) The technical problems the invention addresses are: i. Solar systems require cells to harvest energy. Large-scale solar farms often pose significant environmental hazard, and require real estate to harbor them. ii. Evaporative desalinization plants require significant amounts of energy to produce clean product.

(C) The invention solves both problems by utilizing the solar heat absorbed into surface water. It needs no solar cell, and the real estate needed to found a productive plant is minimal. Instead of distillation it uses crystalline cold purification, which produces energy with its co-process instead of requiring energy input. Co-processes can be utilized to remove organic compounds from the by-product.

(D) The principal use of the invention is to combat anthropopogenic climate change by removing heat from the global system. Further if carbonic compounds are removed oceanic acidification may be reduced.

### CROSS REFERENCE TO RELATED APPLICATIONS

NOT APPLICABLE

### STATEMENT REGARDING FEDERALLY SPONSORED RESEARCH OR DEVELOPMENT

NOT APPLICABLE

### REFERENCE TO A SEQUENCE LISTING, A TABLE OR A COMPUTER PROGRAM LISTING

NOT APPLICABLE

### BACKGROUND OF THE INVENTION

The invention is intended for use in the field of both renewable energy and sustainable desalinated water production. The renewable energy source is solar energy that has been absorbed by the ocean. The energy is transferred via refrigerant to do work. The source of desalinated water is salt water that undergoes phase change following the heat energy being harvested.

Prior cold desalinization was deemed inefficient due to energy required to freeze raw materiel and difficulty separating crystal from brine.

### BRIEF SUMMARY OF INVENTION

The invention and general idea of the process consists of the following: A reversible refrigeration cycle contains the evaporation and condensing coils within the walls of heat transfer tanks. The evaporation tank contains a screw or scrape to remove small ice crystals from the walls. Heat energy enters the refrigerant. Ice crystals are formed and pushed upward. Exiting the tank they are loosened slightly by vibration pushed out and rinsed with clean water at freezing and vacuum filtered. Brine is recovered for processing.

Heat exits the refrigerant into working solution to power turbine or otherwise produce useable energy.

### BRIEF DESCRIPTION OF THE SEVERAL VIEWS OF THE DRAWING

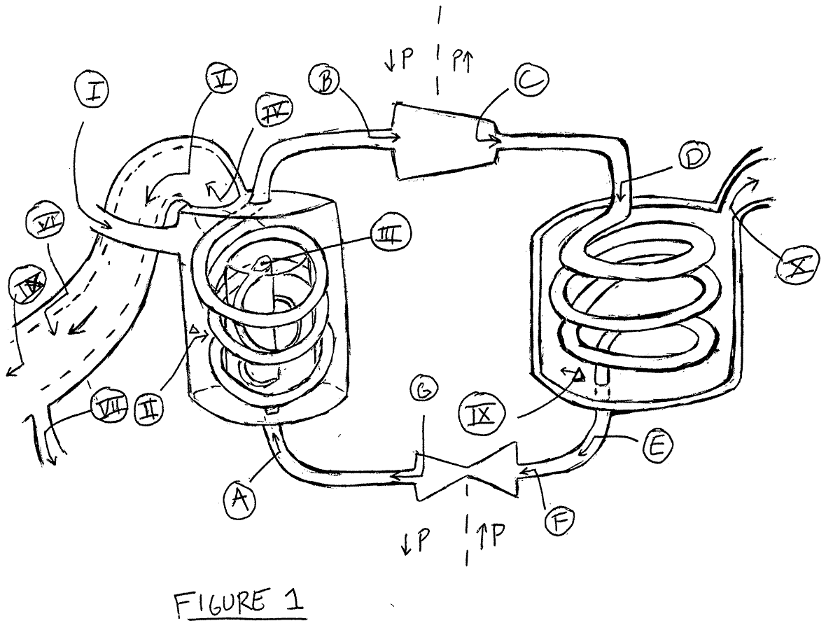

Refers to FIG. 1 replacement drawing—

I. Salt water enters cold tank.
II. Heat energy is absorbed into refrigerant.
III. Heat energy leaves water causing phase change. Water crystals form and are borne upward by screw.
IV. Crystals are vibrated to loosen
V. Brine begins to separate.
VI. Freezing water rinses crystals.
VII. Vacuum filter pulls brine for processing.
IIX. Clean water crystals exit system.
IX. Heat exits refrigerant.
X. Working liquid phase changes to steam to power turbine or is harnessed by other means.
A. Low pressure liquid enters evaporation coils.
B. Low pressure vapor is compressed.
C. High pressure vapor exits compressor.
D. High pressure vapor is condensed.
E. High pressure vapor and liquid exit hot tank.
F. High pressure vapor and liquid expand over valve.
G. Low pressure liquid and vapor exit valve.

### DETAILED DESCRIPTION OF THE INVENTION

The invention is intended to be used in a coastal region. It may be used anywhere there is access to water. It may have applications for industrial cooling towers or any water-cooled process. The invention harnesses solar energy but requires no solar cells to produce clean energy or energy input to produce clean water.

1. The invention would provide clean usable renewable solar energy harvested from surface water and potable water processed simultaneously by a cold crystalline desalinization process.
